(19) United States
(12) Patent Application Publication
(10) Pub. No.: US 2010/0003198 A1
(43) Pub. Date: Jan. 7, 2010

(54) Antimicrobial Topical and Other Preparations Containing Fluorescent or Illumination Dye and Tracer Agents or Compounds

(76) Inventors: Patrick John Stolmeier, San Antonio, TX (US); Jonathan P. Stolmeier, San Antonio, TX (US)

Correspondence Address: Patrick John Stolmeier, 219 E. Craig Place, San Antonio, TX, 78212, US

(21) Appl. No.: 12/498,217

(22) Filed: Jul. 6, 2009

Related U.S. Application Data
(60) Provisional application No. 61/133,936, filed on Jul. 7, 2008.

(51) Int. Cl. A61K 49/00

(52) U.S. Cl. 424/10.3

(57) ABSTRACT

A formulation for topical application to human skin and other surface preparation compositions containing antimicrobial, antifungal and antiviral compounds which remain on human skin and other surface compositions to reduce or dominate bacteria, virus and fungi present, prevent future contamination by their presence and substantive character, contain agents or compounds that contain antibacterial, antiviral and antifungal properties and flourescent dyes and tracers that contain illumination properties exhibiting various colons including clear and no color pigment, that when exposed to certain light such as ultraviolet, will produce an illumination effect in various colors including dear and no color for a period of time within a preferred range of thirty minutes to six hours or longer from the time of initial application, and prior to for instance application of ultraviolet light, such flourescent dyes and tracers appear to be clear in color, invisible or have no color pigment showing on the skin or other surfaces, but when exposed to for instance ultraviolet fight, the surface illuminates allowing a person to determine if human skin or other surface has been adequately covered with the antibacterial, antifungal or antiviral composition agent or compound, and to significantly increase the activity to reduce and eliminate bacteria, virus and fungi on the surface, and support programs for antibacterial, antifungal and antiviral quality control, compliance, verification and education for industry and human handwash technique.

### BACKGROUND OF THE INVENTION

1. Technical Field of the Invention

This invention relates to the preparation of aqueous organic or inorganic formulations containing water soluble or nonwater-soluble antibacterial, antifungal or antiviral agents or compounds, in addition to flourescent dyes and tracers of various colors including clear and no color pigment that produce illumination when exposed to for instance ultraviolet light, for topical application to human skin and other surfaces wherein the formulation reacts on human skin and other surfaces to provide an antimicrobial treatment which is substantive to human skin and other surfaces, retains its antimicrobial activity while present on human skin and other surface, moisturizes human skin by its presence, produces an illumination on human skin and other surface when exposed to light such as ultraviolet, and will illuminate within a preferred range of thirty minutes to six hours or more from the time of initial application, and prior to for instance application of ultraviolet light, such flourescent dyes and tracers appear to be clear or invisible or have no color pigment showing on human skin or other surface, but when exposed to for instance ultraviolet light, the surface illuminates for determining suitable coverage of the formulation on the surface, and supports programs for antimicrobial quality control, compliance, verification and education for industry and human handwash technique, enhanced by the effect of active ingredients such as the antimicrobial agents chloroxylenol, chlorhexidine and their derivatives, metal, metal alloys, metal ions and their derivatives, and contain material with dermal anchoring human skin mechanical properties such as propylene glycol and others known in prior art, and therapeutic and non therapeutic essential oils, synthetic and organic agents, compounds or colors.

2. Background of the Invention

Formulations containing agents or compounds that inhibit or eliminate micro-organisms on human skin and other surfaces have been employed in prior an in hand sanitizers and cleansers, body lotions, body wash, shampoo and other surface applications. Formulations containing flourescent dyes and tracers that once applied to human skin and other surfaces produce an illumination to such surfaces by exposing such surfaces to for example ultraviolet light have been employed in a wide variety of applications in prior art. Various flourescent dyes and tracers of various colors, shades and consistencies exist in prior art and that may or may not be government approved or certified for use on human skin or other surfaces.

For health, safety and economical reasons, it is most desirable to apply such antimicrobial formulations or illumination causing flourescent dyes and tracers from an aqueous medium, which may contain additives, agents and compounds for the purpose for which is to increase stability, provide scent and various colors, improve performance, moisturize human skin, and present various brightness, shades and length of flourescent dye and tracer illumination periods.

The prior art and open literature disclose antimicrobial properties of chloroxylenol, alcohols, ethylenediamine tetra acitic acid, chlorhexidine, triclosan, metal, metal alloys, metal ions and others, and their derivatives against a wide range of pathogens including but not limited to:

Bacteria:

Citrobacter freundii

Citrobacter diversus

Corynebacterium diptheriae

Diplococcus pneumoniae

Micrococcus sp. (I)

Micrococcus sp. (II)

Micrococcus sp. (III)

Mycobacterium spp.

Staphylococcus albus

Staphylococcus aureus

Methicillin resistant staphylococcus aureus (MRSA)

Staphylococcus citrens

Staphylococcus epidermidis

Streptococcus faecalis

Streptococcus pyogenes

Vancomycin resistant enterococcus faecium (VRE)

Acinetobacter buamanni

Acinetobacter calcoaceticus

Campylobacter jejuni

Enterobacter aerogenes

Enterobacter aglomerans (I)

Enterobacter aglomerans (II)

Escherichia coli

Klebsiella pheumoniae

Legionella pneumophila

Listeria monocytogenes

Nisseria gonorrhoeae

Proteus mirabilis

Proteus morganii

Proteus vulgaris

Providencia spp.

Pseudomonas

Pseudomonas aeruginosa

Pseudomonas fragi

Salmonella choleraesuis

Salmonella enteritidis

Salmonella gallinarum

Salmonella paratyphi A

Salmonella schottmuelleri

Salmonella typhimurium

Salmonella typhosa

Serratia marcescens

Shigella flexnerie Type II

Shigella sonnei

Virbrio cholerae

Viruses:

Adenovirus Type IV

C-Difficile

Feline Pneumonitis

Hepatitis B

Hepatitis C

Herpes Simplex Type I & II

HIV-1 (AIDS)

Influenza A (Japan)

Influenza A2 (Aichi)

Influenza A2 (Hong Kong)

Parinfluenza (Sendai)

Parvovirus

Poliovirus

Reovirus

Respiratory Synctia

Rhinovirus 39

Fungi and Mold:

Alternaria alternata

Asperigillus niger

Aureobasidium pullulans

Candida albicans

Cladosporium cladosporioides

Drechslera australiensis

Gliomastix cerealis

Microsporum audouinii

Monilia grisea

Phoma fimeti

Pithomyces chartarum

Scolecobasidium humicola

Trychophyton interdigitale

Trychophyton mentagrophytes

Tuberculociadal

Mycobacterium bouis (TB)

The open literature and the prior art above do not teach topical human skin and other surface application, antimicrobial activity, human and industry qualify control and education benefits of included illumination producing flourescent dyes and tracers of aqueous or non aqueous organic or inorganic solutions such as this invention to human skin or other surfaces, or do they teach such application methods utilizing these agents or compounds as broad spectrum or hospital grade antimicrobial formulations with verifiable assurance of adequate human skin and other surface coverage with these formulations, or do they teach formulations that include flourescent dye and tracer application to human skin and other surfaces wherein illumination from said flourescent dyes and tracers may be formulated and controlled to limit illumination period within a preferred range of thirty minutes to six hours or more, and prior to for instance application of ultraviolet light, such flourescent dyes and tracers appear to be invisible, clear in color or have no color pigment showing on human skin or other surfaces, and after exposure to for instance ultraviolet light, the surface may be illuminated to determine adequate coverage of the formulated solution on human skin or other surfaces, and serve as an industry and human educational tool for proper handwash technique.

In U.S. Pat. Nos. 4,965,063; 5,057,303; 5,064,635; 5,110,492; 5,357,989; WO 92/10148; U.S. Pat. Nos. 5,929,004, and 6,267,976 there are described flourescent or illumination dye compositions that are included in a cleansing solution for the purpose of initially determining surface coverage, but when exposed to air, natural or ultraviolet light, the illuminated color properties vanish to prevent discoloration or staining of the surface, or applied to a surface for magnetic crack testing of metal.

In contradistinction, the present invention is directed for use and preparation of a formulated solution, that when applied to human skin or other surface, has an antimicrobial effect, and when exposed to light such as ultraviolet light, the surface becomes illuminated with a clear or color showing on the surface, otherwise, the surface color shall appear invisible. The illumination of the surface allows the applicant from thirty minutes up to six hours or more to determine whether the surface had been adequately covered with the antimicrobial formulated solution. This invention comprises as a verification and confirmation tool to determine adequate surface coverage of the antimicrobial formulated solution on human skin and other surfaces, and for human and other surface antimicrobial formulated solution compliance, and education for proper handwash technique.

### SUMMARY OF INVENTION

Medical doctors and scientists alike are puzzled to learn that some bacteria, virus and fungi are resistant to antibiotics through various changes or mutation in their cell structures or DNA. Medical, detention and food processing and handling facilities had been the central breeding ground for drug resistant bacteria, virus and fungi. In recent years, these drug resistant organisms have migrated out of these facilities and into the community, infecting otherwise healthy individuals.

The key to preventing medical and other facility and community acquired infection from drug resistant organisms is to prevent or reduce contamination on human skin and other surfaces. Although prior art supports the existence of antimicrobial solutions and cleansers used to eradicate dangerous microbials, none teach or contain a mechanism for assurance that a surface has been adequately covered and cleansed with an antimicrobial formulation or that proper hand or other surface wash technique has been used to eliminate microbials. This invention comprises of formulations containing antimicrobial agents or compound and agents or compounds containing illumination properties that when applied to human skin and other surfaces, adequate surface coverage and surface cleansing can be determined immediately after initial application, as well as confirming or verifying for up to sis hours after initial application that adequate surface coverage and surface cleansing had been performed.

Various aqueous organic or inorganic human skin and other surface formulation applications containing triclosan, chlorhexidine or its derivatives, chloroxylenol or its derivatives, ethylenediamine tetra acitic acid, essential oils, xylitol, cetylpyridinium chloride, alcohol, metal, metal alloys, electrolytically or non electrolytically generated metal ions and combinations thereof suitable as antimicrobial hand sanitizers, body washes, skin cleansers, hand soaps, shampoo, body moisturizing lotions, and other surface disinfectants, cleansers, and sanitizers have been prepared. The formulations disclosed in this invention impart cleansing, antimicrobial effect, moisturizing and illumination on human skin and other surfaces. These formulations, when exposed to light such as ultraviolet produce an illumination on the surface. Otherwise, these formulations on such surfaces remain invisible to the human eye. Prior to application, such illumination flourescent dyes and tracers may appear to be clear in color, contain no color pigment, and invisible.

Prior art has failed to recognize that illumination producing flourescent dyes and tracers can function as an antimicrobial active component when applied to human skin and other surfaces. Prior art has also failed to recognize when illumination producing flourescent dye and tracer formulations are added to agents, compounds and formulations of antimicrobial and dermal anchoring skin mechanical property solutions suitable for topical human skin and other surface applications, such illumination producing flourescent dye and tracer formulations contribute, increase or add to antimicrobial activity of the formulated solution, and present an illumination of the surface when exposed to for instance ultraviolet light, otherwise remain invisible to the human eye, but when exposed for instance to ultraviolet light, allow the applicant of the formulated solution to determine whether human skin or otter surface has been adequately coated or covered to assure maximum benefit of antimicrobial activity upon the surface, and utilize such for antimicrobial human and industrial quality control, verification, compliance and education for proper handwash technique.

Prior art has failed to recognize that flourescent dyes and tracers may be formulated to limit the illumination time period caused by such dyes and tracers within a preferred range of thirty minutes to six hours, or more. These formulations enhance the efficacy of the antimicrobial human and industrial quality control, verification, compliance and education applications of the invention. A formulation produced for the process of treating human skin and other surfaces, wherein the formulation of claim 1, wherein the illumination flourescent or tracer agent is selected from a group of Federal Department of Agriculture. Environmental Protection Agency or Federal Drug Administration suitable dyes with flourescent or tracer illumination properties; and wherein the anti-microbial agent is selected from the group consisting of: if the active ingredient is an anti-microbial, it may be chlorohxylenol or its derivatives, chlorhexidine or its derivatives, such as chlorhexidine gluconate, chlorhexidine digluconate, chlorhexidine diacetate, chlorhexidine dihydrochloride, or it may be selected from the group consisting of nisin, bis-diguanides, polyhexamethylene biguanide (PHMB), benzalkonium chloride (BAC), benzethonium chloride (BZC), methylbenzethonium chloride, triclosan, triclocarban, tribromsalan, amyltricresols, parachlorometaxylenol (PCMX), phenol, iodine, nonylphenoxypoly (ethyleneoxy) ethanoliodine, poloxamer-iodine complex, undecoylium chloride iodine complex, bisquaternary ammonium agents or compounds, plymeric quaternary ammonium agents or compounds, alcohols, cationic peptides, organometaulic antiseptics, alkyl pyridinium salts such as cetyl pyridinium chloride (CPC), essential oils such as tea tree, lavender, thyme, thymol, carvacrol, eugenol, cinnamic, lemon grass, lemon oil, oregano, citrus, bergamot, orange, clove, anise, cinnamon, eucalyptus, spruce, grapefruit, pine, cineole, moroterpenol, liralool oxide, rare crypton, aldehydes, dlimonene, xylitol and cetylpyridinium chloride. Metal alloys such as silver, copper or zinc, electrolytically or non electrolytically generated ions of copper, silver or zinc, and their combinations and derivatives in particle, flake or powder form, essential oils, and their combinations and derivatives; and wherein the anchoring agent is selected from a group consisting of propylene glycol or glycol derivative, wherein various agents or compounds, such as wax, paint, epoxy, laquer, varnish, polyurathane, plastic or polymers are included.

The formulation further comprising an agent selected from a group consisting of deionized water, ethyl alcohol, isopropyl alcohol, alcohol deriviatives, citric acid or its derivatives, triethanolamine, polyacrylic acid, various fragrances, ethylenediamine tetra acitic acid, and octylphenol ethoxalate.

The readers attention is directed to all papers and documents which are referenced herein, filed concurrently with or previous to this specification in connection with this application and which are open to public inspection with this specification, and the contents of all such papers and documents are incorporated herein by reference.

All of the features disclosed in this specification (including any accompanying claims, abstract and drawings), or all of the steps or formulations of any method or process so disclosed may be combined in any combination, except combinations where at least some of such features or steps are mutually exclusive.

Each feature disclosed in this specification (including any accompanying claims, abstract and drawings) may he replaced by alternative features, steps or formulations serving the same, equivalent or similar purpose, unless expressly stated otherwise. Thus, unless expressly stated otherwise, each feature disclosed is only one example of a generic series of equivalent or similar features.

The intention of this invention is not restricted to the details of the foregoing claims. The invention extends to any novel one, or any novel combination of the agents or compounds, formulation or features disclosed in this specification (including any accompanying claims, abstract and drawings) or to any novel one, or any novel combination of the steps or formulations of any method or process so disclosed. For instance, flourescent dyes and tracers may be formulated to cause illumination on human skin or other surface applications for a time period less or more than the preferred thirty minute to six hour or more range, the invention may include formulations that may be less or greater than measured amounts of solutions, agents or compounds listed in the inventions preferred formulation ranges, and flourescent dye and tracer formulations may be included in antimicrobial activity shampoo, skin lotion, skin wash, skin sanitizing, nasal wash and other cleansing solution formulations, as well as various antimicrobial, active and inactive additive agents, compounds and chemicals, flourescent dyes and tracers, both organic and inorganic, and the invention may include various formulations of chemicals, agents or compounds encapsulated together by various polymers, plastics, waxes or materials in a micro-encapsulated time release process, and illumination dye and tracer properties and particle size may be altered or adjusted to regulate the length of illumination period from initial application or exposure, or allow for removal using an aqueous solution.

### Antimicrobial Topical Skin and Other Surface Preparations Containing Flourescent or Illumination Dye and Tracer Agents or Compounds

### Field of Search:

### US Patent References:

U.S. Pat. No. 6,267,976 Apr. 14, 2000 Barnhart et al; An antimicrobial skin cleanser with photosensitive dye used to color the solution allowing a person to see the solution as it is applied to determine whether the surface is covered. When exposed to natural or flourescent light, the dye fades so it does not stain the surface.
U.S. Pat. No. 5,929,004 Oct. 10, 1997 Ushijma et al; Detergent for cleaning tire wheels wherein a color change indicator occurs in the detergent and color fades with exposure to carbon dioxide. Used to determine maximum cleaning effect of the detergent.
U.S. Pat. No. 5,357,989 Mar. 11, 1997 Jun. 25, 1992 Gathani; Dental floss impregnated with a PH sensitive dye producing color change when exposed to an acid. PH is used to detect possible dental canes or a cariogenic environment.
U.S. Pat. Nos. 5,110,492 and 5,064,635 and 5,057,303 and 4,965,063 Aug. 13, 1990 Casey; A cleaning solution with a germicide and dye. When dispensed on a surface, the dye color will show on the surface and then lose color when exposed to the air. Used to confirm that a surface has been initially covered and the dye vanishes to prevent discoloration or staining of the surface.

U.S. Pat. No. 6,626,047 Sep. 30, 2003 Vetterlein, To test cracks on a surface of manufactured parts, an illumination dye is applied, penetrates into a crack and can be visible for magnetic crack, testing.

### Foreign Reference

WO 92/10148 Jun. 25, 1992 Gathani; Dental floss impregnated with a PH sensitive dye producing color change when exposed to an acid. PH is used to detect possible dental caries or cariogenic environment.

In U.S. Pat. Nos. 4,965,063; 5,057,303; 5,064,635; 5,929,004; and 6,267,976 an illumination dye was added to a cleaning solution that may contain a germicide or antimicrobial compound, that after application onto a surface and exposed to natural or ultraviolet light, the illumination effect fades or disappears. These inventions are used to determine initial adequate coverage of the cleaning solution onto a surface, the dye then lades or vanishes to prevent discoloring or staining of the surface.

These inventions fail to teach that after initial application of a cleansing solution containing an illumination dye, that in addition to determining adequate coverage after immediate application, the dye will not fade or vanish for a period of between thirty minutes to six hours, to determine adequate surface coverage for verification and compliance for industry, human health and education for proper hand wash technique. These inventions also fail to teach that the addition of an illumination dye aides the antimicrobial process.

In U.S. Pat. No. 6,626,047 the invention considers use of an illumination dye to cover a surface for magnetic testing for cracks in a surface, including manufactured parts. This invention fails to teach that after initial application of a cleaning solution containing an illumination dye, that in addition to determining adequate coverage after immediate application, the dye will not fade or vanish for a period of between thirty minutes to six hours, to determine adequate surface coverage for verification and compliance for industry, human health and education for proper hand wash technique. This invention also fails to teach the addition of illumination dye aides the antimicrobial process.

In U.S. Pat. No. 5,357,989 and WO 92/10148 the invention considers impregnating an illumination dye into dental floss or fibers for the purpose of detecting possible dental caries or a cariogenic environment. This invention fails to teach that after initial application of a cleaning solution containing an illumination dye, that in addition to determining adequate coverage after immediate application, the dye will not fade or vanish for a period of between thirty minutes to six hours, to determine adequate surface coverage for verification and compliance for industry, human health and education for proper hand wash technique. This invention also fails to teach the addition of illumination dye aides in the antimicrobial process.

### Other References:

Use of a Topical Flourescent Dye to Evaluate Effectiveness of Sunscreen Application, Archives of Dermatology: Vol. 134 No. 4, April 1998: Barry J. Hunt: The Use of Flourescent Dye in Highlighting Some Construction Problems, Structural Survey Vol. 12, Issue: 6 page. 4-7 year: 1994: 1 K J Rothman, S. Greenland, measures of disease frequency, In: K J Rothman, S. Greenland, eds. modern Epidemiology, 2^{nd} ed. Philadelphia: Lippincott, Williams and Wilkins, 1998. pp.-29-46 2. D Pittet, R P Wenzel. Nosocomial bloodstream infections: secular trends, rates, mortality, and contributions to hospital deaths. Arch Intern Med 155: 1177-1184, 1995 3. R W Haley, D H Culver. J W White. The efficacy of infection surveillance and control programs in U.S. hospitals. AMJ Epidemiol 121: 182-205, 1985. 4. D. S. Paulson, Designing a handwash for healthcare workers. Soaps/cosmet/chem spec 6: 12-21, 1986. 5. D. S. Paulson, Efficacy evaluation of a 4% chlorhexidine gluconate as a full-body shower wash. Am. J. Infect Control 21: 205-209, 1993. 6. D. S. Paulson, To glove or wash: a current controversy. Food oval 6: 56-68, 1996. 7. S. Seeburg, A Lindberg, B R Bergman. Pre-operative shower bath with 4% chlorhexidine detergent solution: reduction of Staphylococcus aureus in skin carriers and practical application, In: H Maibach, R Aly, eds. Skin Micro-biology: Relevance to clinical infection. New York: Springer Verlag, 181 pp. 92-97; a Brandberg, I Anderson. Preperative whole body disinfection by shower bath with chlorhexidine soap; effective transmission of bacteria from skin flora. In: H Maibach. R Aly, eds. Skin Micro-biology: Relevant to clinical infection. New York: Springer Verlog, 1981 pp. 92-97: L J Hayek, J M Emerson. Pre-operative whole body disinfection: a controlled clinical study, J. Hosp. Infect II (supplB): 15-19, 1988.

Provisional Patent Application No. 61/133,936 filed in Jul. 7, 2008 is incorporated herein by reference. The Abstract, Claims, Description and Summary of invention and statements contained in Provisional Patent Application No. 61/133,936 are incorporated in this Non-Provisional Patent Application and it is intended that this Non-Provisional Patent Application claims the benefit of Provisional Application No. 61/133,936.

What is claimed is:

1. An illumination or tracer formulation, wherein said illumination or tracer agent or compound is an antibacterial, antifungal or antiviral topical human skin or other surface formulation comprising of an antimicrobial agent selected from a group of chloroxylenol, chlorhexidine, alcohol, iodine, triclosan and their derivatives, metal alloys or ions to include copper, zinc and silver and their derivatives, other antimicrobial compounds and their derivatives known in poor art and agents or compounds known in prior art as compatible with use of antimicrobial topical human skin or other surface agents or compounds wherein such agents or compounds are included with flourescent dye or tracer agents or compounds with illumination properties known in prior art when exposed to light such as ultraviolet, the solution will produce an illumination effect, otherwise, the dye will appear to have no color properties and invisible.

2. An illumination or tracer formulation according to claim 1, wherein said illumination or tracer agent or compound is 005 to 5.0% of an illumination flourescent or tracer agent or compound, wherein the illumination or tracer agent or compound is selected from a group of Federal Department of Agriculture, Environment Protection Agency or Federal Drug Administration approved dyes with illumination or tracer properties such as FD&C flourescent Dye (DNC) (MX659 Pylam-Cert Fluorescent or Distyrl Bipheryl), and Synthetic Organic Colorant of various colors, textures and consistencies, in the form of a powder, liquid or gel.

3. An illumination or tracer formulation according to claim 1, wherein said illumination or tracer agent or compound is the illumination flourescent or tracer agent or compound particles or molecules that are of different size wherein the longevity or duration of the illumination or tracer effect can be regulated and measured from 30 minutes to six hours, or longer.

4. An illumination or tracer formulation according to claim 1, wherein said illumination or tracer agent or compound is 0.005 to 5.0% of an illumination flourescent or tracer agent or compound, wherein the illumination or tracer agent or compound has an anti-microbial effect.

5. An illumination or tracer formulation according to claim 1, wherein said illumination or tracer agent or compound is the illumination flourescent or tracer agent or compound, when applied to a surface such as human skin or other surface such as metal, plastic, rubber, fabric, nylon, wood, leather, vinyl, ceramics, composites, derivatives or combinations thereof and other surfaces known in prior art, appear to have no visible color, color pigment, illumination or tracer properties.

6. An illumination or tracer formulation according to claim 1, wherein said illumination or tracer compound is the illumination flourescent or tracer agent or compound, when applied to a surface such as human skin, or other surface such as metal, plastic, rubber, fabric, nylon, wood, leather, vinyl, ceramics, composites, derivatives or combinations thereof and other surfaces, known in prior art, appear to have no visible color, color pigment, illumination or tracer properties until exposed to light, such as ultra violet, the illumination flourescent or tracer properties are activated and visible in a color such as green, blue, yellow, orange, white or clear.

7. An illumination or tracer formulation according to claim 1, wherein said illumination or tracer agent or compound is a flourescent or illumination tracer agent or compound wherein the longevity or duration of the illumination period may be regulated from thirty minutes to six hours or more by adding various chemicals, agents or compounds into for instance a micro-capsule, and for such micro-capsule to erode, dissolve or rupture and release its contents by effect such as friction, time release, temperature, and mix with an illumination flourescent or a tracer agent or compound.

8. The formulation according to claim 1, wherein the active ingredient is an antibacterial, antifungal or antiviral agent or compound.

9. The formulation according to claim 1, wherein an anchoring agent or compound consists of various percentages of an agent or compound such as selected from a group of diisopropyl adipate, diisobutyl adipate, higher fatty acid ployoxethylene glycerol and polyglycerol or essential oils.

10. The formulation according to claim 1, wherein the antibacterial agent or compound is chloroxylenol or a chloroxylenol derivative.

11. The formulation according to claim 1, wherein a polyol is selected from a group consisting of propylene glycol or a glycol derivative.

12. The formulation according to claim 1, wherein the antibacterial, antifungal or antiviral agent or compound is selected from the group consisting of chlorhexidine or its derivatives, such as chlorhexidine gluconate, chlorhexidine digluconate, chlorhexidine diacetate, chlorhexidine dihydrochloride, or it may be selected from the group consisting of nisin, bis-diguanides, polyhexamethylene biguanide (PHMB), benzalkonium chloride (BAC), benzethonium chloride (BZC), methylbenzethonium chloride, triclosan, triclocarban, tribromsalan, amyltricresols, parachlorometaxylenol (PCMX), phenol, iodine, nonylphenoxypoly (ethyleneoxy) ethanoliodine, poloxamer-iodine complex, undecoylium chloride iodine complex, bisquaternary ammnium compounds, plymeric quaternary ammonium compounds, alcohols, cationic peptides, organometaulic antiseptics, alkyl pyridinium salts such as cetyl pyridinium chloride (CPC), metal alloys such as silver, copper or zinc, their ions and combinations and derivatives generated electrolytically or non electrolytically, in particle, flake or powder form, essential oils, and their combinations and derivatives; and wherein the anchoring agent is selected from the group consisting of propylene glycol or glycol derivative.

13. The formulation according to claim 1, wherein the anchoring agent or compound consisting of propylene glycol or glycol derivative is 0.25 to 5.0%.

14. The formulation according to claim 1, further comprising an agent or compound selected from a group consisting of deionized water, ethyl alcohol, isopropyl alcohol, geraniol, inalool, thujanoi, myrcenol, terpineol, menthol, piperitol, alcohol derivatives, citric acid or its derivatives, triethanolamine, polyacrylic acid, various fragrances, ethylenediamine tetra acid or octylphenol ethoxalate.

15. The formulation according to claim 1, wherein agents or compounds, and material known in prior art that are compatible with use of antibacterial, antiviral and antifungal agents and compounds known in the prior art selected from a group of wax, paint, epoxy, lacquer, polyurethane, varnish, plastic and polymers.

16. A formulation according to claim 1, wherein the antibacterial, antifungal and antiviral agent or compound is selected from a group of therapeutic, non therapeutic or synthetic essential oils and their derivatives, organic agents and their derivatives, such as tea tree, lavender, thyme, thymol, carvacrol, eugenol, cinnamic, lemon grass, lemon oil, oregano, citrus, bergamot, orange, clove, anise, cinnamon, eucalyptus, spruce, grapefruit pine, cineole, moroterpenol, liralool oxide, rare crypton, aldehydes and dlimonene.

17. A formulation of reducing or eliminating the number of micro-organisms on the surface of human skin, hair, nails and other surfaces such as metal, metal alloys, plastic, rubber, ceramics, fabrics, nylon, wood, leather, composites thereof and other surfaces known in prior art. Said formulation comprising of a topical human skin and other surface sanitizing and cleansing solution, liquid, lotion, wash, soap, antiseptic, disinfectant, applied to human skin or other surface, to a towelette or surface liquid cleanser and disinfectant comprised of an aqueous solution of water-soluble or nonwater-soluble organic or inorganic agents or compounds in an amount effective to eliminate or inhibit the growth of nitro-organisms, comprised of an illumination flourescent dye or tracer agent or compound of various colors including clear and no color pigment, capable of producing antimicrobial activity and an illumination effect on human skin and other surface that once exposed to light, such as ultraviolet will cause an illumination of the surface, otherwise the illumination compound appears invisible, and having referred formulations of:
a. Deionized Water 70-85%
Ethyl Alcohol 0.1-90.0%
Propylene Glycol 0.25-5.0%
Triethanolamine 0.59%
Polyacrylic Acid 0.43%
Chloroxylenol 0.1-5.0%
FD&C Fluorescent Dye (DNC) or a Synthetic
Organic Colorant 0.005-5.0%
(MX659 Pylam-Cert Flourescent) (Day-Glo DG-00, A-594-5)
Fragrance 0.20%
b. Deionized Water 70-85%
Isopropyl Alcohol 0.1-90.0%
Propylene Glycol 0.25-5.0%
Triethanolamine 0.59%
Polyacrylic Acid 0.43%
Chloroxylenol 0.1-5.0%
FD&C Flourescent Dye (DNC) or a Synthetic
Organic Colorant 0.005-5.0%
(MX659 Pylam-cert Flourescent) (Day-Glo DG-00, A-594-A)
Fragrance 0.20%
c. Deionized Water 80-99%
Isopropyl Alcohol 0.1-90.0%
Distyrl Biphenyl (Tenapol) 0.005-5.0%
Ethylenediamine Tetra Acitic Acid (EDTA) 0.14-5.4%
Propylene Glycol 0.25-5.0%
Octylphenol Ethoxalate 2.00%
Chloroxylenol 0.1-5.0%
Fragrance 0.20%
d. Deionized Water 80-99%
Ethyl Alcohol 0.1-90.0%
Distyrl Biphenyl (Tenapol) 0.005-5.0%
Ethylenediamine Tetra Acitic Acid (EDTA) 0.14-5.4%
Propylene Glycol 0.25-5.0%
Octylphenol Ethoxalate 2.00%
Chloroxylenol 0.1-5.0%
Fragrance 0.20%
e. Deionized Water 80-99%
Isopropyl Alcohol 0.1-90.0%
FD&C Fluorescent Dye (DNC) or Synthetic
Organic Colorant 0.005-5.0%
Ethylenediamine Tetra Acitic Acid (EDTA) 0.14-5.4%
Propylene Glycol 0.25-5.0%
Octylphenol Ethoxalate 2.00%
Chloroxylenol 0.1-5.0%
Fragrance 0.20%
f. Deionized Water 80-99%
Ethyl Alcohol 0.1-90.0%
FD&C Fluorescent Dye (DNC) or Synthetic
Organic Colorant 0.005-5.0%
Ethylenediamine Tetra Acitic Acid (EDTA) 0.14-5.4%
Propylene Glycol 0.25-5.0%
Octylphenol Ethoxalate 2.00%
Chloroxylenol 0.1-5.0%
Fragrance 0.20%

18. Formulations contained in a thru f above may include one or more of the following agents or compounds, derivatives of each, combinations, and in various amounts.
a. Deionized Water
Glycerin
Glyceryl Stearate PEG-100 Stearate
Isopropyl Mysistate (IPM)
Chlorhexidine
Triclosan
Iodine
Cetylpyridinium chloride
Xylitol
Copper
Zinc
Silver
Electrolytically or non electrolytically generated copper, silver and zinc ions Synthetic Organic Colorant of various colors, textures, particles and forms (liquid, gel and powder)
Essential oils of various types, odors, mixtures, therapeutic or non therapeutic or synthetic essential oils and their derivatives, organic agents and their derivatives such as tea tree, lavender, thyme, thymol, carvacrol, eugenol, cinnamic, lemon grass, lemon oil oregano, citrus, bergamot, orange, clove, anise, cinnamon, eucalyptus, spruce, grapefruit, pine, cineole, moroterpenol, liralool oxide, rare crypton, aldehydes and dlimonene, wintergreen, spearmint, along with sorbitol, polysorbates, propylene glycol, tochopherol, various alcohols and sodium chloride.

19. A formulation for a human skin sanitizing and cleansing solution, lotion, wash, shampoo and other surface liquid cleanser and disinfectant comprised of an aqueous solution of water-soluble or nonwater-soluble organic or inorganic agents or compounds in an amount effective to eliminate or inhibit the growth of micro-organisms, comprised of an illumination flourescent dye or tracer agent or compound of various colors including clear and no color pigment, capable of producing antimicrobial activity and an illumination effect upon human skin and other surface, when exposed to light such as ultraviolet, wherein the illumination upon human skin or other surface will dissipate and cease to illuminate within a preferred range of thirty-minutes to six hours or more from the time of initial application, and prior to for instance application of ultraviolet light, such flourescent dyes and tracers appear to be clear or invisible or have no color pigment showing on human skin or other surface, but when expose to for instance ultraviolet light, the surface becomes illuminated.

20. A formulation to control, set or stage the time period for when the illumination producing dyes or tracers cease to produce their illumination properties by for instance adding various formulations of chemicals, agents or compounds that when exposed to various illumination producing dyes or tracers they terminate in whole or in part, the illumination producing properties of such dyes or tracers on human skin and other surfaces. Such chemicals, agents or compounds may be combined into a micro-capsule, and adding that micro-capsule into the formulations referred herein, and for such micro-capsule to erode, dissolve, rupture or release its contents into the formulations by such effect such as friction, various periods of time, various temperature or environmental condition or effect known in prior art, as time release or micro encapsulation processes.
